(19) United States
(12) Patent Application Publication
(10) Pub. No.: US 2002/0083951 A1
(43) Pub. Date: Jul. 4, 2002

(54) Implantatble identification marker

(76) Inventors: Peter Stegmaier, Ponte Capriasca (CH); Arie Bruinink, Effretikon (CH); Viola Schlosser, Pfaffikon (CH); Ruedi Gwalter, Jona (CH); Jorg Mayer, Niederlenz (CH)

Correspondence Address: Harness, Dickey & Pierce, P.L.C., P.O. Box 828, Bloomfield Hills, MI, 48303, US

(21) Appl. No.: 10/028,753

(22) Filed: Dec. 20, 2001

(30) Foreign Application Priority Data
Dec. 27, 2000 (EP) 00811245.0

(51) Int. Cl. A61B 19/00

(52) U.S. Cl. 128/899; 128/903

(57) ABSTRACT

An implantable identification marker comprises an electronic device (4) enclosed within a vial (1; 2) isolating the device (4) from body fluids of the animal after insertion of the marker into the body of an animal. The marker further comprises a fixation structure (8; 5) providing a significantly in-creased total surface of the marker and/or a reduced specific weight of the marker. This improves the integration of the marker into the surrounding tissue avoiding migration within the animal.

### FIELD OF THE INVENTION

The invention relates, in general, to an identification marker, and more particularly to an implantable identification marker having a biocompatible covering.

### BACKGROUND OF THE INVENTION

One of the major requirements associated with manufacture of an implantable electronic transponder is the encapsulation of the IC circuit hybrid assembly and antenna coil, so that after insertion into the animal these critical components are isolated from animal body fluids. The encapsulation material must be bio-compatible and completely non-adverse to the surrounding tissue at the implant site.

It is further known, that a certain percentage of such identification markers migrate away from the initial location throughout the lifetime of the animal. It is, however, preferred to have a stationary identification marker. First of all, the reading area should be always the same within each individual animal. Furthermore, such an identification marker may migrate into areas where it hinders the animal.

U.S. Pat. No. 5,074,318 discloses an anti-migration means in the form of a polymer layer coating with a high coefficient of friction. Also polypropylene is used as a coating. Furthermore the layer can be formed through partially inserting a marker in a mold cavity, injecting resin and curing it. Finally it is suggested to etch an outer glass coating of the marker.

U.S. Pat. No. 5,840,148 discloses a bio-compatible anti-migration cap with two sharp projections withholding the marker in the injection device and preventing migration against the direction of insertion of the marker, i.e. the loss of the marker through the insertion wound. However, U.S. Pat. No. 5,840,148, may prevent migration in one direction only and the effects of the coatings in U.S. Pat. No. 5,074,318 may not be sufficient.

Therefore, it is necessary to provide an implantable identification marker that is biocompatible as well as includes an anti-migration feature to prevent movement of the marker within the subcutaneous region of the animal or object in which the marker has been inserted.

### SUMMARY OF THE INVENTION

The identification marker of the present invention is to be implanted subcutaneously, i.e. under the skin. It comprises an electronic device, e.g. a transponder. The transponder can containing a variety of information including identification information about an animal that can be read by an external detector. Such implantable transponder are known and have significant utility in the biomedical field as well as for identification of domestic animals as e.g. dogs or cats.

The identification marker of the present invention further provides an anti-migration or fixation structure for stabilizing the identification marker within the subcutaneous region. It is therefore an object of the invention to improve the integration of the marker into the surrounding tissue avoiding migration within the animal.

The advantage of the marker according the invention is the possibility of interaction of the tissue surrounding the marker with the structure of the marker. This interaction is fundamentally different from the anti-migration devices presently known in the art and previously described, such as the etched glass surface, the layer coating, the cap or the pointed unidirectional projections.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a schematic sectional view of a marker according to a first embodiment of the invention in its implanted state,

FIG. 2 is a view form above onto a marker constructed in accordance to a second embodiment of the invention,

FIG. 3 is a sectional view taken along line 2-2 in FIG. 2,

FIG. 4 is a sectional view of a marker according to a third embodiment of the invention before implantation,

FIG. 5 is a sectional view of a portion of a marker according to a fourth embodiment of the invention after its implantation,

FIG. 6 is a schematic sectional view of a marker according to a fifth embodiment of the invention in its implanted state, and

FIG. 7 is a schematic sectional view of an implantation set with a marker according to FIG. 1.

### DETAILED DESCRIPTION OF THE PREFERRED EMBODIMENTS

FIG. 1 shows a schematic sectional view of a marker according to a first embodiment of the invention after insertion into an animal. Usually said markers are used for domestic animals as dogs or cats. However, it can also be used with other animals.

The marker according to FIG. 1 is composed of a bio-compatible glass vial 1 having a wall 2. The initially open glass vial 1 may be filled with a potting material 3 up to a predetermined level. Then a transponder unit 4 is introduced into the glass vial 1 and into the potting material 3. The transponder 4 can be comprised of an IC circuit and an antenna coil. It is also possible that the transponder 4 is an electronic identification device having others and/or additional functionalities.

Subsequently, the glass vial 1 is sealed. This can be performed through closing the open vial 1 with a cap, using a flame-based technology or with the use of a laser. The material 3 may also be a UV curable material in a liquid state.

Beside the use of a one-piece vial 1 as shown in FIG. 1, other embodiments may use a cap to build the gas impermeable means to protect the transponder from the environment. The transponder unit 4 is shown as box shaped.

The vial 1 is enclosed within a fine net 5, embroidery, knit or wickerwork. Although the textile net 5 may be of any material, it can be made of polypropylene filaments. Preferably the net 5 is tube-shaped with a diameter smaller then the diameter of the vial 1, so that it is biased against said vial 1.

At least at one end 6 or at both ends 6 and 16 of the vial 1 the net 5 merges into an implantation structure 7. In FIG. 1 two implantation structures 7 and 17 are provided at first and second 6 and 16 of the vial 1. Every structure 7 or 17 is preferably composed of a restricted structure 10 welded to the net 5 in order to ensure that the vial 1 stays encapsulated within the net 5. Furthermore the structures 7 or 17 comprise a multitude of single filaments 8 of a bio-compatible material, forming a cone 9. The single filaments 8 have a length of e.g. 3 to 10 millimeters with a typical length of the vial 1 of 1 to 2 centimeters. Within the cone 9, the single filaments 8, multi-filaments, textiles, ribbons, net, knit or wickerwork may have a distance one from another between 10 and 1000 micrometers. The number of filaments 8 may be between 20 and 200 and they may have a diameter between 10 and 100 micrometers. Within the body of an animal the filaments 8 of such a marker may permit blood vessels and tissue to grow into the region of the cone 9 and to encapsulate each single filament 8 to ensure the stable positioning of the implanted marker. The filaments 8 may be more or less straight (as shown in FIG. 1) or the may be curled and entwine.

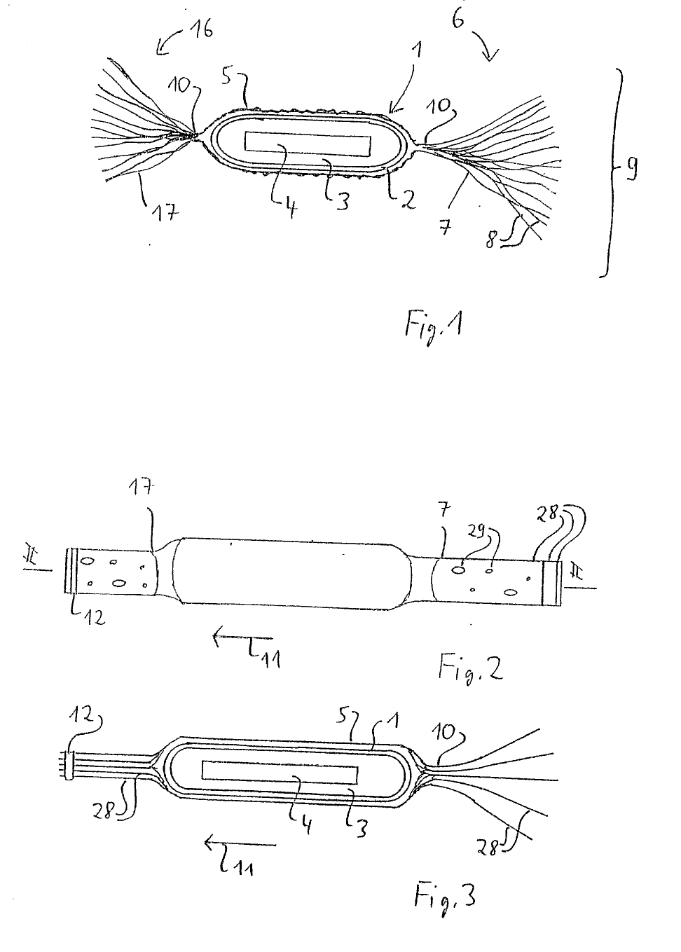

The marker to be implanted may have the single filaments 8 encapsulated within a rapidly biodegradable material, in a way that the single filaments 8 do not extend beyond the diameter of the vial 1. After the dissolution of said material, the pre-biased single filaments 8 spread and form the cone 9. It is also possible to provide a single band 12 (see FIGS. 2 and 3) of biodegradable material around the ends of the single filaments 8 to ensure that the diameter of the bound single filaments 8 does not surpass the diameter of vial 1 (including the net 5). It also is possible to permit the single filaments 8 at the distal end 6 of the vial 1 to spread within the injection needle to ensure the position of the marker within said needle before insertion of the marker into the animal body. Arrow 11 shows the direction of implantation in FIGS. 2 and 3.

FIG. 2 shows a schematic view from above onto a marker constructed in accordance to a second embodiment of the invention. Here the end structures 7 or 17 are composed of several sheets 28 of bio-compatible material. It can be seen from the upper-most sheet 28, shown in FIG. 2, that they contain holes 29 of different diameters. The diameter of these holes 29 can vary e.g. between 2 and 400 micrometers. Greater diameters may depend on material choice.

FIG. 3 shows a sectional view taken along line 2-2 in FIG. 2. It can be seen that there are several sheets 28, glued together in the zone 10. The number of five sheets 28 is chosen to simplify the graphical representation. Usually there are between 2 and 20 sheets 28. The sheets 28 may be pre-biased to spread after insertion of the marker into the animal. The length of the sheets 28 may be equal to the above mentioned length of the single filaments 8 of FIG. 1. The distance between two sheets 28 may vary between 10 and 1000 micrometers. Therefore the embodiment according to FIG. 3 shows the same properties as the marker according to FIG. 1. Sheets 28 may be bound together with a single band 12 of biodegradable material or glued together with such material.

FIG. 4 shows a sectional view of a marker according to a third embodiment of the invention before implantation. The marker comprises a compressible element 38 along the vial 1 in a zone 26 and in the portions 7 beyond the vial 1. The compressible element can be formed of a variety materials, more preferably foam. This compressible element 38, which may be provided only along the wall 2 of the vial 1 without extending any further or extending into both portions 7 and 17, can be encapsulated with a thin biodegradable membrane 40. Within the fabrication procedure of the marker the gas between vial 1 and membrane 40 can be evacuated and therefore the compressible element 38 can be compressed to e.g. one fifth to one tenth of the expanded volume. After insertion of this marker in the form shown in FIG. 4 into an animal, the membrane 40 is dissolved and the element 38 expands. This permits the surrounding tissue to grow into the structures of element 38. Furthermore the larger volume of the foam 38 reduces the specific weight of the marker towards and even below the specific weight of the surrounding tissue, so that gravitational migration effects can be avoided.

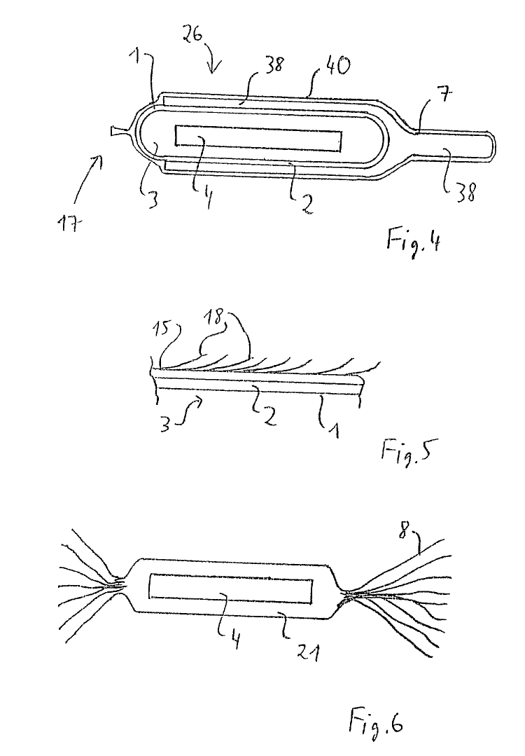

FIG. 5 shows a sectional view of a portion of a marker according to a fourth embodiment of the invention after its implantation. The wall 2 of the vial 1 is surrounded by a planiforme structure 15 possessing filaments 18 extending away from the wall 2 of the vial 1. The filaments 18 of FIG. 5 may all be orientated in one direction. They may have a length of e.g. 500 to 5000 micrometers and may be of the same material as the filaments of the first embodiment of the invention. The planiforme structure 15 surrounding the vial 1 comprises a multitude of parallel rows with similar series of filaments 18 which are orientated in opposite direction in every row. This ensures the fixation of the vial 1 in the tissue by virtue of the growth of material into the space between the different filaments 18, into the space between filaments 18 in one row and into the space between filaments 18 of different rows.

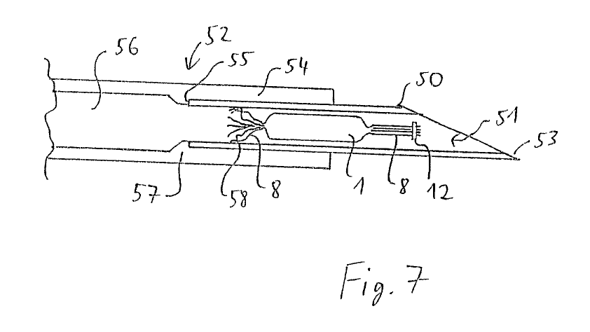

FIG. 6 shows a schematic sectional view of a marker according to a fifth embodiment of the invention in its implanted state. The embodiment uses filaments 8 as in FIG. 1. The difference between the two embodiments lies in the fact that in FIG. 6 the transponder 4 is surrounded directly by the material 21 or layered sets of material forming the filamentous tails. Said vial 1 can be omitted, if the enclosing material constitutes a reliable barrier for water vapour, i.e. the material has no water vapour permeability. This approach can be used in all shown embodiments.

FIG. 7 shows a schematic sectional view of an implantation set with a marker according to FIG. 1. The implantation set is a needle assembly formed from a stainless steel hollow tube 50 having an exit opening 51 and an entrance opening 52. Exit opening 51 is formed in the shape of an inclined edge forming a sharp point 53 permitting the tube 50 to easily penetrate an animal's skin. Tube 50 is molded into a sleeve 54 and abuts against shoulder 55 having the same inner diameter as the hollow tube 50. In the area 56 of the sleeve beyond the hollow tube 50 the inner diameter is greater then the inner diameter of the tube 50. A tapered zone 57 forms the transitional area between the area 56 and the tube 50.

FIG. 7 shows a marker according to FIG. 3 disposed within the tube 50. The single filaments 8 of the marker directed to the exit opening 51 are bound with a single band 12 of biodegradable material to ensure that the diameter of the bound single filaments 8 does not surpass the diameter of vial 1 of the marker. The filaments 8 positioned on the other side of the vial 1 are free and can extend beyond the inner diameter of the tube 50. Therefore, they are in contact with said tube 50 at points 58 and interference fit with the inside diameter of tube 50 and prevent the displacement of the marker during storage or transport. A plunger (not shown) is slideably disposed inside the sleeve 54 and pushes the marker outside the exit opening 51 and injects it into the animal. This plunger may also be used to push the marker, preloaded in the sleeve 54, into the smaller tube 50. The tapered portion 57 between sleeve 54 and tube 50 is especially useful, when the marker is manufactured according to an embodiment according to FIGS. 4 or 5.

It can be appreciated that the anti-migration or fixation structure provides a significant increase of the surface area surrounding the marker after its implantation. This is achieved through providing at least one zone 8, 18, 28, 38 with significantly increased total surface area. This may also lead to an overall decrease of specific weight. This can be a cone 9 of single filaments 8, a foam 38 or expanding sheets 28. These zones can be provided at one 6 or both ends 16 of the marker, they can also be provided alongside 26 the transponder 4. In the latter case the zone is preferably initially (before implantation) compressed, since the diameter of the marker upon insertion into the animal is preferably as small as possible. The compression may be achieved through encapsulating it with a biodegradable membrane and subsequent application of a vacuum upon fabrication of the marker. This enlarges the radius of the marker by generally less than 200 micrometers. In the structures beyond the ends 6 and 16 of the transponder 4 the zones 7, 17 with significantly reduced specific weight can be pre-biased and encapsulated in a biodegradable material.

It is also possible to exert radially compressing forces upon the marker encapsulated with the material of significantly reduced specific weight to enter it into the implantation needle according to FIG. 7. Then the implantation needle with the marker is packaged. Said compressed material will ensure that the marker does not slide out of the hollow needle 50 without the action of a plunger.

The property of the zones of significantly reduced specific weight is accompanied by the surface increase within theses zones and therefore greater possibilities of interaction between the surrounding tissue and the marker. This surface increase can especially be provided through woven, knitted, braided, non-woven and stamped fabric structures. In the case that a vial 1 of a different material as biocompatible glass is used, it may be encapsulated by the knit, net or wickerwork tube-shaped element 5. This tube 5 is then sealed at both ends 6 an 16 of the marker by making use of a glue, pressure, heat or ultrasonic welding to create the restriction 10.

The description of the invention is merely exemplary in nature and, thus, variations that do not depart from the gist of the invention are intended to be within the scope of the invention. Such variations are not to be regarded as a departure from the spirit and scope of the invention.

This application hereby incorporates by reference the disclosure in European patent application 00811245.0 filed Dec. 27, 2000.

1. An implantable identification marker comprising:
an electronic device enclosed within a biocompatible material isolating the device from body fluids of the animal after insertion of the marker into the body of an animal; and
a fixation structure attached to the biocompatible material thereby providing a significantly increased total surface of the marker.

2. The implantable identification marker according to claim 1, wherein the electronic device is a transponder.

3. The implantable identification marker according to claim 1, wherein the fixation structure extends radially from the biocompatible material thereby permitting tissue elements of the animal to grow into said fixation structure following insertion.

4. The implantable identification marker according to claim 1, wherein said biocompatible material isolating the device has a first end and a second end.

5. The implantable identification marker according to claim 4, wherein the fixation structure extends beyond said first end of said biocompatible material.

6. The implantable identification marker according to claim 4, wherein the fixation structure extends beyond said first end and said second end of said biocompatible material.

7. The implantable identification marker according to claim 1, wherein the fixation structure further comprises a net biased against the biocompatible material and forming at least one implantable structure composed of a restricted structure to ensure that the biocompatible material is encapsulated within the net.

8. The implantable identification marker according to claim 7, wherein the fixation structure further comprises a plurality of filaments.

9. The implantable identification marker according to claim 8, wherein said filaments for a cone-shaped structure.

10. The implantable identification marker according to claim 7, wherein the fixation structure comprises a plurality of sheets.

11. The implantable identification marker according to claim 10, wherein said filaments further comprise a plurality of holes.

12. The implantable identification marker according to claim 11 wherein said holes have the same diameter.

13. The implantable identification marker according to claim 11 wherein said holes have different diameters.

14. The implantable identification marker according to claim 1, wherein the biocompatible material isolating the electronic device and the fixation structure form one piece.

15. The implantable identification marker according to claim 1 wherein the fixation structure further comprises a compressible element disposed adjacent to the biocompatible material.

16. The implantable identification marker according to claim 1 wherein a biodegradable membrane is disposed adjacent to the fixation structure.

17. The implantable identification marker according to claim 16 wherein the biodegradable membrane encapsulates the fixation structure.

18. The implantable identification marker according to claim 1 further comprising an implantation set consisting of a needle portion to be inserted into the skin of an animal, wherein the needle portion is hollow.

19. The implantable identification marker according to claim 18 wherein the needle portion has an exit opening having a first inner diameter and an entrance opening having a second inner diameter, larger then the first inner diameter, thereby permitting the loading of the marker in the needle.

20. The implantable identification marker according to claim 19, wherein the fixation structure of the marker holds the marker within the hollow needle portion, so that the marker can only be displaced in the needle portion through action of a plunger pushing the marker through the exit opening of the needle portion.

21. An implantable identification marker comprising:
an electronic device enclosed within a biocompatible material isolating the device from body fluids of the animal after insertion of the marker into the body of an animal; and
a fixation structure attached to the biocompatible material thereby providing a reduced specific weight of the marker.

22. A method for implanting an identification marker subcutaneously within an animal comprising the steps of:
obtaining an electronic device encoded with identifying information for an animal;
enclosing the electronic device with biocompatible material to form a vial;
covering said vial with a fixation structure to form an identification marker;
compressing at least a part of the fixation structure;
injecting the marker into the animal; and
expanding the fixation structure within the animal to reduce migration of the marker.

23. The method of claim 22 wherein the fixation structure is held in compression with a biodegradable member which dissolves after injection thereby allowing the fixation structure to expand and engage tissues in the subcutaneous layer of the animal.

24. The method of claim 22 further comprising:
enclosing the vial with a net having filaments therein, portions of the filaments being bound by biodegradable material which expands after injection of the marker within the animal.

25. The method of claim 22 which further comprises:
placing the identification marker within an implantation device containing a hollow needle portion and a plunger; and
injecting the identification marker into the animal by slideably disposing the identification marker through the needle portion and into a subcutaneous skin layer of the animal by the use of a plunger, wherein the fixation structure secures itself within the subcutaneous layer of the animal preventing the migration of the marker.
